(19) United States
(12) Patent Application Publication
(10) Pub. No.: US 2024/0391774 A1
(43) Pub. Date: Nov. 28, 2024

(54) METHOD FOR PRODUCING ULTRA-PURE BIS(CHLOROSULFONYL)IMIDE

(71) Applicant: SPECIALTY OPERATIONS FRANCE, Lyon (FR)

(72) Inventor: Etienne Schmitt, Soucieu-En-Jarrest (FR)

(73) Assignee: SPECIALTY OPERATIONS FRANCE, Lyon (FR)

(21) Appl. No.: 18/694,120

(22) PCT Filed: Sep. 21, 2022
(86) PCT No.: PCT/EP2022/076166

§ 371 Date: Mar. 21, 2024

(30) Foreign Application Priority Data
Sep. 23, 2021 (EP) 21315166.5

(51) Int. Cl. C01B 21/093

(52) U.S. Cl. CPC C01B 21/0935; C01P 2002/88; C01P 2006/80

(57) ABSTRACT

The present invention relates to a process for manufacturing a bis(chlorosulfonyl)imide (HCSI) of ultra-pure (UP) grade with a purity of at least 99.0 mol. % with respect to the total number of moles of HCSI. In addition, the present invention relates to an HCSI of UP grade obtainable from the process, and to the use of the HCSI of UP grade for preparing a lithium bis(fluorosulfonyl)imide (LiFSI). The present invention also relates to a process for manufacturing a LiFSI comprising the preparation of an HCSI of UP grade according to the present process. The present invention relates to a composition comprising a LiFSI with a purity of at least 99.99 mol. % with respect to the total number of moles of LiFSI in the composition, and to the use of a composition comprising a LiFSI obtainable from the present process in a lithium-ion secondary battery.

### CROSS-REFERENCE TO RELATED PATENT APPLICATIONS

This application claims priority filed on 23 Sep. 2021 in EUROPE with Nr. 21315166.5, the whole content of this application being incorporated herein by reference for all purposes.

### TECHNICAL FIELD

The present invention relates to a process for manufacturing a bis(chlorosulfonyl)imide (HCSI) of ultra-pure (UP) grade with a purity of at least 99.0 mol. % with respect to the total number of moles of HCSI. In addition, the present invention relates to an HCSI of UP grade obtainable from the process, and to the use of the HCSI of UP grade for preparing a lithium bis(fluorosulfonyl)imide (LiFSI). The present invention also relates to a process for manufacturing a LiFSI comprising the preparation of an HCSI of UP grade by a process according to the present invention. The present invention relates to a composition comprising a LiFSI with a purity of at least 99.99 mol. % with respect to the total number of moles of LiFSI in the composition, and to use of the LiFSI obtainable from the present process in a lithium-ion secondary battery.

### BACKGROUND OF THE INVENTION

For more than several decades, lithium secondary batteries including lithium-ion batteries have retained a dominant position in the market of rechargeable energy storage devices thanks to their many benefits comprising light-weight, reasonable energy density and good cycle life.

Nonetheless, current lithium secondary batteries still suffer from relatively low energy densities with respect to the required energy density, which keeps increasing for high power applications such as electrical vehicles (EVs), hybrid electrical vehicles (HEVs), grid energy storage, etc.

Accordingly, the electrolytes with high purity are more and more required to obtain higher-power batteries, because they make it possible to increase the nominal voltage of lithium-ion batteries. Notably, the impurities in salts and/or electrolytes may impact the overall performance and stability of the lithium-ion batteries in a negative manner that the identification and quantification of impurities in salts and/or electrolytes and the understanding of their working mechanisms on battery performances have been continuously of high interest in battery fields. In particular, various approaches have been investigated to develop salts and/or electrolytes having minimum amounts of impurities with a very low residual moisture content.

In the field of lithium-ion batteries, LiPF₆ has been extensively used thanks to its high solubility in non-aqueous polar solvents, notably organic carbonates, despite other drawbacks such as relatively poor thermal stability and high sensitivity toward water. Consequently, bis(fluorosulfonyl)imide salts, in particular LiFSI, have attracted remarkable attention from the battery players as a promising candidate to replace LiPF₆ thanks to its excellent ionic conductivity and good resistance to hydrolysis. Under this context, different processes, reactants and intermediates leading to LiFSI have been described in the literature.

Considering that LiFSI is intended to be used in a lithium-ion secondary battery and that the impurities present in the LiFSI may induce the reduction of performances and stability of the resulting lithium-ion battery, it is critical to limit the impurities present in the LiFSI to an amount as low as possible.

Most of the existing processes for manufacturing LiFSI contain numerous steps, with the consequence that many by-products or other contaminants, e.g., residual organic solvents, moisture, etc., are inevitably generated. The removal of these by-products and/or contaminants is costly and time-consuming, leading to reduced yield and purity of the final LiFSI. In some cases, the purification methods are hardly scalable to an industrial level, and cause a poor environmental footprint for the corresponding process.

EP3381923 B1 (CLS Inc. and Solvay Fluoro GmbH) relates to a process for producing LiFSI, particularly by using HCSI to be reacted with anhydrous ammonium fluoride having a water content of 0.01 to 3,000 ppm as a fluorinating reagent, and then directly treated with an alkaline reagent without further purification.

A common LiFSI purification step mostly comprises at least one liquid/liquid extraction technique to separate an aqueous phase and an organic phase, where the selection of solvents to be used is critical. However, the extraction always accompanies several drawbacks. For instance, multiple extraction steps to obtain optimal output are often necessary that large volume of organic solvents are inherently required, which will eventually result in the increase of its processing/recycling cost.

US2019/0292053A1 (Arkema) describes a process for manufacturing a LiFSI comprising a reduced content of water and sulfates, which has been dried and purified by means of drying and purification steps, the drying step being notably performed by using a short-path thin-film evaporator under particular conditions to remove the solvents used without degradation of the target product, i.e., LiFSI. Nonetheless, thusly-prepared LiFSI salt still contains certain amount of impurities including Cl−, SO₄²⁻, F⁻, FSO₃Li⁻, CO₃²⁻, ClO₃⁻, ClO₄⁻, NO₂⁻, NO₃⁻, etc.

The concentration of LiFSI is rather difficult because heating LiFSI at high temperature and/or for a long time induces the decrease of the product yield and purity, resulting in high production cost by following additional multiple purification steps, especially in presence of organic solvents (and/or other contaminants). Moreover, the boiling point of the reaction solvent increases because alkali metal salt of bis(fluorosulfonyl)imide form and also because the solvation between LiFSI and solvent molecules easily occurs.

U.S. Pat. No. 9,985,317 B2 (Nippon Shokubai) relates to an alkali metal of fluorosulfonylimide having good heat resistance with a reduced content of specific impurities and water content, and also to a process for producing an alkali metal salt of fluorosulfonylimide, which is capable of easily removing solvents from a reaction solution by bubbling a gas into the reaction solution containing the alkali metal salt of fluorosulfonylimide and/or by concentrating the solution of the alkali metal salt of fluorosulfonylimide by thin-layer distillation.

Overall, the complexity of LiFSI manufacturing processes, including several time-consuming and costly purification steps, is generally caused by the occurrence of side-reactions generated in the course of the manufacturing processes, and by the necessity to remove these formed by-products by means of purification steps and/or drying steps. This complexity should still be addressed in order to provide LiFSI having superior heat resistance and electrochemical performances. In a nutshell, there still exists a need for a novel process for preparing a LiFSI having minimum amount of impurities and a very low residual moisture content, which can be more easily scalable for industrialization in a reasonably economical manner.

One of the known intermediates leading to LiFSI is HCSI, which is usually isolated after its synthesis by means of classical batch or semi-batch distillation technique.

WO2015/004220 (Lonza Ltd.) relates to a method for the preparation of bis(halidesulfonyl)imide compounds, notably bis(chlorosulfonyl)imide in a continuous mode via three consecutive steps at elevated temperatures, in comparison with a batch reaction according to the conventional methods.

Even though many prior art documents describe HCSI intermediates as pure materials, concrete evidence of the exact purity/yield is mostly missing or with single analytical results being provided without any absolute reference of the HCSI material for comparison. It is consequently difficult to distinguish the quality of HCSI employed in the various LiFSI manufacturing processes using HCSI as a raw material. Hence, the reported yields for HCSI cannot be considered as accurate in absence of a quantitative analytical method providing a strong evidence for its purity.

As a key raw material for many LiFSI manufacturing processes, the quality of HCSI has obviously a strong impact on the generation of undesired by-products in the course of HCSI-based LiFSI manufacturing processes, and consequently accessing an HCSI having an exceptionally high purity as a key intermediate is a strong advantage.

Under these circumstances, the present inventor intensively studied and found an optimal process to obtain a higher-purity HCSI under milder conditions with a comparable yield, with which eventually a higher-purity LiFSI can be obtained with reduced efforts for purification, while lessening the environmental impact of the resulting LiFSI manufacturing process. It was also identified that by applying suitable continuous distillation conditions, a higher-purity HCSI can be obtained under reduced thermal stress.

### SUMMARY OF THE INVENTION

A first object of the present invention is a process for manufacturing a bis(chlorosulfonyl)imide (HCSI) of ultra-pure (UP) grade comprising the steps of:
(i) providing a crude HCSI mixture (I) comprising HCSI, heavy fractions and light fractions;
(ii) removing the light fractions from the crude HSCI mixture (I) so as to obtain a HCSI mixture (II);
(iii) transferring the HCSI mixture (II) to a thin-film evaporator; and
(iv) distillating the HCSI mixture (II) to isolate the HCSI of UP grade,

wherein the HCSI of UP grade presents a purity of at least 99.0 mol. % with respect to the total number of moles of HCSI, as determined by differential scanning calorimetry (DSC) according to ASTM E928-19.

A second object of the present invention is an HCSI of UP grade obtainable from the process as described above.

A third object of the present invention is the use of an HCSI of UP grade obtainable from the process as described above for preparing a lithium bis(fluorosulfonyl)imide (LiFSI).

A fourth object of the present invention is a process for manufacturing a lithium bis(fluorosulfonyl)imide (LiFSI), comprising the preparation of an HCSI of UP grade by the process as described above.

A fifth object of the present invention is a composition comprising a LiFSI with a purity of at least 99.99 mol. % with respect to the total number of moles of LiFSI in the composition, and the remainder being water, residual raw materials and impurities comprising F⁻, Cl⁻, SO₄²⁻ and FSO₃⁻.

A sixth object of the present invention is the use of the LiFSI obtainable by the process as described above in a lithium-ion secondary battery.

It was surprisingly found by the inventor that the HCSI of UP grade manufactured according to the process of the present invention results in an increased performance in the subsequent steps to produce LiFSI, for instance a fluorination step to produce a crude NH₄FSI, which will result in the high yield and purity of LiFSI to be produced as final product via a lithiation step. In addition, the inventor also found that using the HCSI of UP grade to synthesize LiFSI reduces the need for purification and creates a positive impact on the impurity profile of the final LiFSI without compromising the yield. Moreover, the heavy fractions can be used again in a subsequent distillation to recover the HCSI, i.e., no yield drop occurs from the process according to the present invention.

### BRIEF DESCRIPTION OF DRAWINGS

FIG. 1 describes DSC curves of HCSI of UP grade after WFSP distillation, wherein the 4^{th} melting peak is integrated, and the 3^{rd} crystallization peak is visible on top.

FIG. 2 shows comparison of DSC results between the HCSI of UP grade (indicated as solid lines with 24 cumulative cycles) and the HCSI distilled in batch (indicated as dotted lines with 4 cumulative cycles).

FIG. 3 describes DSC curves of the HCSI after batch distillation, followed by WFSP distillation, wherein the 4th melting peak is integrated, and the 3rd crystallization peak is visible on top. The HCSI of UP grade was not obtained by this approach.

### DETAILED DESCRIPTION OF THE INVENTION

### Definitions

Throughout this specification, unless the context requires otherwise, the word “comprise” or “include”, or variations such as “comprises”, “comprising”, “includes”, including” will be understood to imply the inclusion of a stated element or method step or group of elements or method steps, but not the exclusion of any other element or method step or group of elements or method steps. According to preferred embodiments, the word “comprise” and “include”, and their variations mean “consist exclusively of”.

As used in this specification, the singular forms “a”, “an” and “the” include plural aspects unless the context clearly dictates otherwise. The term “and/or” includes the meanings “and”, “or” and also all the other possible combinations of the elements connected to this term.

The term “between” should be understood as being inclusive of the limits.

In the present application, any description, even though described in relation to a specific embodiment, is applicable to and interchangeable with other embodiments of the present disclosure. Furthermore, where an element or component is said to be included in and/or selected from a list of recited elements or components, it should be understood that in related embodiments explicitly contemplated here, the element or component can also be any one of the individual recited elements or components, or can also be selected from a group consisting of any two or more of the explicitly listed elements or components; any element or component recited in a list of elements or components may be omitted from such list. Additionally, any recitation herein of numerical ranges by endpoints includes all numbers subsumed within the recited ranges as well as the endpoints of the range and equivalents.

In the present invention, the term “batch process” is intended to denote a process, where all reactants are fed into the reactor at the beginning of the process and the products are removed when the reaction is complete. No reactant is fed into the reactor and no product is removed during the process.

In the present invention, the term “semi-batch process” is intended to denote a process, which allows the additional feeding of reactants and/or the removal of products in time.

In the present invention, the term “ppm” is intended to denote one part per one million (1,000,000) parts, i.e., 10⁻⁶.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature range of about 120° C. to about 150° C. should be interpreted to include not only the explicitly recited limits of about 120° C. to about 150° C., but also to include sub-ranges, such as 125° C. to 145° C., 130° C. to 150° C., and so forth, as well as individual amounts, including fractional amounts, within the specified ranges, such as 122.2° C., 140.6° C., and 141.3° C., for example.

Unless otherwise specified, in the context of the present invention the amount of a component in a composition is indicated either as the ratio between the weight of the component and the total weight of the composition multiplied by 100, i.e., % by weight (wt. %) or as the ratio between the volume of the component and the total volume of the composition multiplied by 100, i.e., % by volume (vol. %). It is to be understood that both the foregoing general description and the following detailed description are exemplary and are intended to provide further explanation of the invention as claimed. Accordingly, various changes and modifications described herein will be apparent to those skilled in the art. Moreover, descriptions of well-known functions and constructions may be omitted for clarity and conciseness.

A first object of the present invention relates to a process for manufacturing a bis(chlorosulfonyl)imide (HCSI) of UP grade comprising the steps of:
(i) providing a crude HCSI mixture (I) comprising HCSI, heavy fractions and light fractions;
(ii) removing the light fractions from the crude HCSI mixture (I) so as to obtain a HCSI mixture (II);
(iii) transferring the HCSI mixture (II) to a thin-film evaporator; and
(iv) distillating the HCSI mixture (II) to isolate the HCSI of UP grade,

wherein the HCSI of UP grade presents a purity of at least 99.0 mol. % with respect to the total number of moles of HCSI, as determined by differential scanning calorimetry (DSC) according to ASTM E928-19.

In particular, the present inventor found that the light fractions should be removed first from the crude HCSI mixture (I) so as to obtain a HCSI mixture (II), before transferring the HCSI mixture (II) to a thin-film evaporator, to produce the HCSI of UP grade through distillation. In comparison, applying a thin-film evaporator without removing the light fractions from the crude HCSI mixture (I) didn't result in the HCSI of UP grade under the same conditions. In addition, the HCSI mixture (II) should be transferred to a thin-film evaporator, i.e., after the HCSI mixture (II) is obtained from step (ii). The present inventor found that in case the HCSI mixture (II) follows additional distillations in batch, instead of its transfer to a thin-film evaporator, traces of light fractions become still present even after step (ii) due to the extended time during the batch distillations causing thermal degradation, which results in a mixture of traces of light fractions, heavy fractions and HCSI. Such a mixture comprising traces of light fractions in addition to the heavy fractions and HCSI, resulted in less molar purity of HCSI even after the distillation via a thin-film evaporator, because the thin-film evaporator, notably WFSP is more effective in separating a mixture of two compounds.

In one embodiment, the process for manufacturing an HCSI of UP grade is implemented in a sequential order, i.e., from step (i) to step (iv), wherein the sequential order from step (i) to step (iv) can be performed in a successive way or in a stepwise manner.

In the other embodiment, the HCSI mixture (II) is transferred to a distillation boiler before transferring the same in melted form to a thin-film evaporator.

In the context of the present invention, the HCSI used in the process of the present invention may be produced by a known method, for example:
by reacting chlorosulfonyl isocyanate (ClSO₂NCO) with chlorosulfonic acid (ClSO₂OH);
by reacting cyanogen chloride (CNCl), sulfuric anhydride (SO₃) and chlorosulfonic acid (ClSO₂OH);
by reacting sulfamic acid (NH₂SO₂OH), thionyl chloride (SOCl₂) and chlorosulfonic acid (ClSO₂OH).

In a particular embodiment, HCSI is prepared either by the so-called isocyanate route or by the sulfamic route.

In one embodiment, the reaction mixture is produced by reacting chlorosulfonic acid (ClSO₂OH) and chlorosulfonyl isocyanate (ClSO₂NCO). According to this embodiment, step (i) consists in providing a crude HCSI mixture (I) comprising HCSI, heavy fractions and light fractions, wherein such crude HCSI mixture (I) is obtained by reacting chlorosulfonyl isocyanate (ClSO₂NCO) with chlorosulfonic acid (ClSO₂OH).

In another embodiment, the reaction mixture is produced by reacting sulfamic acid (NH₂SO₂OH), chlorosulfonic acid (ClSO₂OH) and thionyl chloride (SOCl₂). According to this embodiment, step (i) consists in providing a crude HCSI mixture (I) comprising HCSI, heavy fractions and light fractions, wherein such crude HCSI mixture (I) is obtained by reacting sulfamic acid (NH₂SO₂OH), chlorosulfonic acid (ClSO₂OH) and thionyl chloride (SOCl₂).

In the other embodiment, the crude HCSI is produced by reacting cyanogen chloride CNCl with sulfuric anhydride (SO₃) and chlorosulfonic acid (ClSO₂OH). According to this embodiment, step (i) consists in providing a crude HCSI mixture (I) comprising HCSI, heavy fractions and light fractions, wherein such crude HCSI mixture (I) is obtained by reacting cyanogen chloride CNCl with sulfuric anhydride (SO₃) and chlorosulfonic acid (ClSO₂OH).

The process of the present invention also applies to commercially available HCSI, in particular if such commercially available HCSI does not present the expected purity. In this embodiment, step (i) may be defined as consisting in providing a “crude HCSI mixture (I)”, comprising HCSI, heavy fractions and light fractions.

In some embodiments, step (ii) consists in heating the HCSI mixture (I) above 40° C. in order for the light fractions to be removed in the form of a gas from the rest of the mixture. In preferred embodiments, step (ii) is conducted at a temperature ranging from 40° C. to 150° C., preferably from 60° C. to 120° C., and more preferably from 90° C. to 120° C.

In some embodiments, step (ii) is performed at atmospheric pressure, or under reduced pressure. In particular embodiments, step (ii) is performed under the pressure of less than 500 mbar abs., preferably less than 200 mbar abs., more preferably less than 100 mbar abs., and even more preferably less than 10 mbar abs.

In some embodiments, the HCSI mixture (II) comprising HCSI and heavy fractions is transferred to a distillation boiler or a transitory vessel before transferring the same to a thin-film evaporator, i.e., before step (iii), but without additional distillation(s) in batch.

In one embodiment, step (iii) is performed at a temperature ranging from 40° C. to 150° C., preferably from 40° C. to 120° C., more preferably from 40° C. to 100° C., even more preferably from 40° C. to 80° C., and most preferably from 40° C. to 70° C.

In one preferred embodiment, the HCSI mixture (II) is maintained in a melted form by heating at temperature range of from 40 to 70° C. during the transition phase. In another preferred embodiment, in case of solidification, the intermediate or final product, i.e., HCSI mixture (II) or HCSI of UP grade, is melted by heating at temperature range of from 40 to 70° C. until complete melting without significant impact on the quality of the final product, i.e., HCSI of UP grade.

In one embodiment, step (iii) is performed at atmospheric pressure, or under reduced pressure. In a preferred embodiment, step (iii) is performed at atmospheric pressure.

In the present invention, the term “thin-film evaporator”, also known as “thin-layer evaporator”, is intended to denote a device used to purify temperature-sensitive products by evaporation enabling short residence time, which allows processing of many heat sensitive and difficult to distill products. Other terminologies can also be used, such as falling film evaporators, rising film evaporators, wiped film evaporators, short-path evaporators, flash evaporators, agitated thin film evaporators, wiped-film short path (WFSP) evaporators, etc.

In one embodiment, the thin-film evaporator is a short-path thin-film evaporator, a WFSP evaporator (with external condenser), or a falling-film evaporator. Such evaporators generate vapors during the evaporation covering a short path, i.e., travelling a short distance, before being condensed in the condenser.

Typically, the short-path thin-film evaporators comprise a condenser for the solvent vapors inside the device, while other types of thin-film evaporators, which are not short-path evaporators, have a condenser outside the device.

In a short-path thin-film evaporator, a thin-film of a product to be distilled is formed on a hot inner surface of the evaporator by continuously applying the product to be distilled on its inner surface. In one embodiment, the short-path thin-film evaporator is equipped with a cylindrical heated body and an (axial) rotor which helps to evenly distribute the product as a thin film to be distilled over the evaporator's inner surface. As the product spirals down the wall, the high rotor tip speed generates highly turbulent flow resulting in the formation of waves and creating optimal heat flux and mass transfer conditions. Subsequently, volatile components are quickly evaporated via conductive heat transfer and the vapors are ready for the condensation, while non-volatile components are discharged at the outlet. One of main problems which can arise during evaporation is fouling that occurs when hard deposits form on the surfaces of the heating medium in the evaporators. Such kind of unfavorable phenomenon can be minimized by continuous agitation and mixing, correlating with a sufficient flow rate of the crude mixture to form a stable film. This sufficient flow rate is defined depending on the type and size of thin-film evaporator to be employed. For example, a flow rate of about 120-125 g/hr is sufficient to obtain a stable film in case of a KD1-type thin-film evaporator commercially available from UIC GmbH.

In the present invention, the term “residence time” is intended to denote the time which elapses between the entry of the remaining reaction mixture into the evaporator and the exit of the first drop of the solution from the evaporator.

The compatibility with a thin-film evaporator largely depends on the properties of the product, in particular the thermal stability of the product to be purified.

The process according to the present invention is advantageous for the main reason that a HCSI of UP grade can be obtained after a distillation phase under milder conditions with a shortened time duration. Usually, after the reaction step where the reaction temperature ranges from 120° C. to 140° C. for a period of from 15 to 25 hours in order to generate an HCSI crude mixture, the HCSI distillation phase requires a temperature range of 100° C. to 145° C. for a prolonged period, possibly ranging from several hours in a laboratory scale to more than 20 hours at an industrial scale. The combination of both reaction and distillation phases causes a cumulated period of thermal stress for the HCSI ranging from about 35 to 45 hours or even more, and causes a substantial color change of the reaction mixture, evolving from colorless to clear yellow, often up to brown, indicating a substantial formation of non-valorizable heavy by-products. By using the process according to the present invention, however, the inventor made it possible to lower the temperature and to reduce the residence time of the distillation phase in a substantial manner, while reducing the overall thermal stress of the thermally-sensitive HCSI.

In a particular embodiment, the distillation step (iv) is implemented under the temperature of 100° C. or less, preferably 90° C. or less, more preferably 80° C. or less, and even more preferably 70° C. or less.

In another particular embodiment, the distillation step (iv) is implemented under the pressure of 10 mbar abs. or less, preferably 5 mbar abs. or less, more preferably 3 mbar abs. or less, and even more preferably 0.5 mbar abs. or less.

In other particular embodiment, the residence time in the distillation step (iv) is 5 minutes or less, preferably 3 minutes or less, more preferably 1 minute or less, and even more preferably for 30 seconds or less.

In a preferred embodiment, the distillation step (iv) is implemented in a short-path thin-film evaporator under a temperature varying from 80° C. to 100° C. and/or a pressure varying from 0.1 to 10 mbar abs. with a residence time of 30 seconds or less.

In the present invention, the purity of the HCSI of UP grade obtained after step (iv) is assessed and more precisely is measured via differential scanning calorimetry (DSC) according to ASTM E928-19. A particular sampling protocol as well as a defined temperature profile, is applied, as described in the experimental section, in order to minimize or completely avoid any decomposition, which may happen during characterization.

In a particular embodiment, the onset temperature is 34° C. or more; the peak temperature is 38° C. or more; the temperature of fusion is 37.5° C. or more. In the other particular embodiment, the normalized integral ranges from about −58 J/g to about −65 J/g. In another particular embodiment, the apex temperature of crystallization peak is 20° C. or more.

In a preferred embodiment, the HCSI of UP grade presents a purity of at least 99.3 mol. % with respect to the total number of moles of HCSI, as determined by DSC according to ASTM E928-19.

In a more preferred embodiment, the HCSI of UP grade presents a purity of at least 99.5 mol. % with respect to the total number of moles of HCSI, as determined by DSC according to ASTM E928-19.

In an even more preferred embodiment, the HCSI of UP grade presents a purity of at least 99.7 mol. % with respect to the total number of moles of HCSI, as determined by DSC according to ASTM E928-19.

In a most preferred embodiment, the HCSI of UP grade presents a purity of at least 99.9 mol. % with respect to the total number of moles of HCSI, as determined by DSC according to ASTM E928-19.

The present inventor also found that the light fractions should be removed first from the reaction mixture, before transferring the crude HCSI and the heavy fractions to a thin-film evaporator, to produce the HCSI of UP grade. In comparison, applying a thin-film evaporator without removing the light fractions from the reactor didn't result in the HCSI of UP grade under the same conditions, most probably due to the reduced number of theoretical plates offered by such a distillation equipment in comparison with more separative types of distillation equipment known from the skilled person. Additionally, applying a thin-film evaporator to an HCSI, previously distilled in batch, didn't result in the HCSI of UP grade under the same conditions.

In the present invention, the expression “light fractions” is intended to denote fractions obtained by distilling the crude HCSI mixture resulting from the reaction phase by applying distillation conditions described for step (iii), either in a batch mode, in a semi-batch mode or in continuous mode.

Non-limitative examples of components from the light fractions comprise chlorosulfonic acid, chlorosulfonyl isocyanate, and/or thionyl chloride, which remain unreacted after the reaction.

In the present invention, the expression “heavy fractions” is intended to denote fractions obtained after distilling the HCSI from the crude mixture (preliminary separated from its light fractions) by applying distillation conditions as described for step (v), either in a batch mode, in a semi-batch mode or in a continuous mode.

Non-limitative examples of components from the heavy fractions comprise residual un-distilled HCSI and related by-products including dimers, trimers and other oligomers which may form from the HCSI and other reaction materials via hydrolysis or other side reactions. The heavy fractions are difficult to valorize and have often to be treated as corrosive chemical wastes in the end.

A second object of the invention is an HCSI of UP grade, which may be obtained from the process as described above.

A third object of the present invention is the use of the HCSI of UP grade, which may be obtained from the process as described above, for preparing a LiFSI

A fourth object of the present invention is a process for manufacturing a lithium bis(fluorosulfonyl)imide (LiFSI), comprising the preparation of an HCSI of UP grade by the process as described above.

In one embodiment, the process for manufacturing a LiFSI comprises the sequential steps of:
(i) providing an HCSI of UP grade obtained by the process as described above;

(ii) fluorinating the HCSI of UP grade with a fluorinating agent to form an ammonium bis(fluorosulfonyl)imide (NH₄FSI); and
(iii) optionally purifying the NH₄FSI obtained from the step (ii); and
(iv) lithiating the NH₄FSI, possibly in a form of a solvate with at least one solvent S₂, with a lithiating agent to form a LiFSI.
   In some embodiments, the NH₄FSI of step (iv) is in the form of a solvate, possibly in a crystallized form, comprising:
50 to 98 wt. %. of the NH₄FSI salt, and
2 to 50 wt. %, of solvent S₂, which is selected from the group consisting of cyclic and acyclic ethers.
   Preferably, the NH₄FSI solvate comprises from 51 to 90 wt. %, more preferably from 78 to 83 wt. % of the NH₄FSI salt.
   Preferably, the NH₄FSI solvate comprises from 10 to 49 wt. %, more preferably from 17 to 22 wt. % of solvent S₂.
   In some embodiments, step (iii) of the above-mentioned LiFSI preparation process comprises:
(iii₁) dissolving the NH₄FSI from step (ii) in at least one solvent S₁;
(iii₂) crystallizing NH₄FSI from step (iii₁) by means of at least one solvent S₂; and
(iii₃) separating the NH₄FSI salt from at least part of the solvents S₁ and S₂, preferably by filtration, to prepare a NH₄FSI solvate.

According to these embodiments, the NH₄FSI from step (ii) may comprise 80 to 97 wt. % of the salt of NH₄FSI, preferably 85-95 wt. %, more preferably 90-95 wt. % by weight, the remaining being impurities.

In step (ii) the fluorination agent is preferably a lithium compound, more preferably selected from the group consisting of lithium hydroxide LiOH, lithium hydroxide hydrate LiOH·H₂O, lithium carbonate Li₂CO₃, lithium hydrogen carbonate LiHCO₃, lithium chloride LiCl, lithium fluoride LiF, alkoxide compounds such as CH₃OLi and EtOLi, alkyl lithium compounds such as EtLi, BuLi and t-BuLi, lithium acetate CH₃COOLi, and lithium oxalate Li₂C₂O₄, more preferably LiOH. H₂O or Li₂CO₃.

The solvent S₁ is preferably selected from the group consisting of acetonitrile, valeronitrile, adiponitrile, benzonitrile, methanol, ethanol, 1-propanol, 2-propanol, 2,2,2,-trifluoroethanol, n-butyl acetate, isopropyl acetate, and mixtures thereof; preferably 2,2,2,-trifluoroethanol.

The solvent S₂ is preferably selected from the group consisting of diethylether, diisopropylether, methyl-t-butylether, dimethoxymethane, 1,2-dimethoxyethane, tetrahydrofuran, 2-methyltetrahydrofuran, 1,3-dioxane, 4-methyl-1,3-dioxane, and 1,4-dioxane, and mixtures thereof; more preferably from the list consisting of diethyl ether, diisopropyl ether, methyl t-butyl ether, 1,2-dimethoxyethane, tetrahydrofuran, 2-methyltetrahydrofuran, dioxane and mixtures thereof; even more preferably being 1,3-dioxane or 1,4-dioxane.

In some preferred embodiments, the fluorinating agent of step (ii) is added to the NH₄FSI over a time range of from about 0.5 hr to about 10 hr.

In another embodiment, the process for manufacturing a LiFSI comprises the sequential steps of:
(i) providing an HCSI of UP grade by the process as described above;
(ii) neutralizing HCSI of UP grade by using an onium halide having water content of 500 ppm or less, preferably 400 ppm or less, and more preferably 300 ppm or less to form the ammonium bis(chlorosulfonyl)imide (NH₄CSI);
(iii) fluorinating the NH₄CSI with a fluorinating agent to form an ammonium bis(fluorosulfonyl)imide (NH₄FSI);
(iv) optionally purifying NH₄FSI obtained from the step (iii); and
(v) lithiating the NH₄FSI with a fluorinating agent to form a LiFSI.

In the other embodiment, the process for manufacturing a LiFSI comprises the sequential steps of:
(i) providing an HCSI of UP grade by the process as described above;
(ii) lithiating the HCSI of UP grade with a lithiating agent to form a lithium bis(chlorosulfonyl)imide (LiCSI);
(iii) optionally purifying the LiCSI obtained from the step (ii); and
(iv) fluorinating the LiCSI with a fluorinating agent to form a LiFSI

In a particular embodiment, the lithiating agent is a lithium halide comprising LiF, LiCl, LiBr and LiI.

In another particular embodiment, the lithiating agent is LiOH, LiOH. H₂O or LiNH₂.

In the other particular embodiment, the fluorinating agent is HF, NH₄F. (HF) n (n=0 to 10), NaF, KF, CsF, AgF, LiBF₄, NaBF₄, KBF₄, or AgBF₄.

In a preferred embodiment, the fluorinating agent is HF.

In another preferred embodiment, the fluorinating agent is NH₄F.

A fifth object of the invention is a composition comprising a LiFSI with a purity of at least 99.99 mol. % with respect to the total number of moles of LiFSI in the composition. The remainder may be residual raw materials or by-products, comprising impurities, such as, F⁻, Cl⁻, SO₄²⁻, and FSO₃⁻, water and residual solvent.

In a preferred embodiment, a composition comprises a LiFSI with a purity of at least 99.99 mol. % with respect to the total number of moles of LiFSI in the composition, and the remainder being residual raw materials or by-products.

In one embodiment, the content of impurities is 50 ppm or less with respect to the total weight of the composition.

In a preferred embodiment, the content of water and impurities is 20 ppm or less with respect to the total weight of the composition

In a more preferred embodiment, the content of water and impurities is 10 ppm or less with respect to the total weight of the composition.

In a particularly preferred embodiment, a composition comprises a LiFSI with a purity of at least 99.99 mol. % with respect to the total number of moles of LiFSI, wherein the composition is in the form of solid.

In another particularly preferred embodiment, a composition comprises a LiFSI with a purity of at least 99.99 mol. % with respect to the total number of moles of LiFSI, wherein the composition is in the form of solution with organic solvents, for instance organic carbonates.

In a more particularly preferred embodiment, a composition comprises a LiFSI with a purity of at least 99.99 mol. % with respect to the total number of moles of LiFSI, wherein the composition is in the form of solution with ethyl methyl carbonate (EMC).

The present invention also relates to the use of the LiFSI obtainable by the process as described above in a lithium-ion secondary battery.

Should the disclosure of any patents, patent applications, and publications which are incorporated herein by reference conflict with the description of the present application to the extent that it may render a term unclear, the present description shall take precedence.

The invention will be now explained in more detail with reference to the following examples, whose purpose is merely illustrative and is not intended to limit the scope of the invention.

### Raw Materials and Device

Chlorosulfonyl isocyanate (ClSO₂NCO): commercially available from Lonza Ltd. or synthesized internally within Solvay.

Chlorosulfonic acid (ClSO₃H): commercially available from Sigma Aldrich

Sulfamic acid (NH₂SO₃H): commercially available from Sigma Aldrich.

Thionyl chloride (SOCl₂): commercially available from Sigma Aldrich

Ammonium chloride (NH₄Cl): commercially available from Sigma Aldrich

Ammonium fluoride (NH₄F): commercially available from Sigma Aldrich

Ethyl methyl carbonate (EMC): commercially available from Sigma Aldrich

Lithium hydroxide monohydrate (LiOH. H₂O): commercially available from Sigma Aldrich

Short-path thin-film evaporator: KD1, commercially available from UIC GmbH.

### Testing Methods

Differential Scanning calorimetry (DSC): For the purity determination by a DSC, ASTM E928-19 was followed with certain optimization of the conditions for the measurement. HCSI sampling must be carried out under strictly inert atmosphere using stainless steel or gold-coated pressure-tight crucibles. DSC is performed with the samples in the range of from 10 to 30 mg. The melting peak obtained after at least two melting/crystallization cycles, and possibly up to 4 cycles, is integrated by the DSC software. As an example, the DSC method used was defined as follows: One cycle from −30° C. to 150° C. (4 melting/3 crystallizations) at 5° C./min. under N₂ gas stream 50 mL/min (duration 4 hours and 12 minutes). As another example, the DSC apparatus from Mettler Toledo was used for the analytical development, where the software commanding the device and performing the data analysis was the STARe software, Version 11.00a (Build 4393), also from Mettler Toledo. Other DSC apparatus can be employed similarly. The crucibles and membranes used for the HCSI DSC analysis can be chosen from a variety of references, including the following ones from Mettler Toledo:
HP Steel crucibles: 51140404
HP Gold-coated crucibles: 51140405
Gold-coated single-use membrane: 51140403

The molar purity can be estimated by means of the “Purity” or “Purity Plus” functions of the software, applying the Van′t Hoff law equation, known from the skilled. DSC purity determination can be looked on as a super melting point determination. DSC purity determination is based on the fact that the impurities lower the melting point of a eutectic system. This effect is described by the Van't Hoff equation, as described by the DSC device supplier in its website: https://www.mt.com/de/en/home/supportive_content/matchar_apps/MatChar_UC101. html.

$${T}_{f} = {T}_{0} - \frac{{RT}_{0} {T}_{fus}}{Δ {H}_{f}} ln ( 1 - {x}_{2.} \frac{1}{F} )$$

The simplified equation is:

$${T}_{f} = {T}_{0} - \frac{{RT}_{0}^{2}}{Δ {H}_{f}} {x}_{2.} \frac{1}{F} ,$$

where T_{f} is the melting temperature (which, during melting, follows the liquidous temperature); T₀ is the melting point of the pure substance; R is the gas constant; ΔH_{f} is the molar heat of fusion (calculated from the peak area); x_{2.0} is the concentration (mole fraction of impurity to be determined); T_{fus} is the clear melting point of the impure substance; F is the fraction melted, and ln is the natural logarithm. In both cases, the reciprocal of the fraction melted (1/F) is given by the equation:

$$\frac{1}{F} = \frac{{A}_{tot} + c}{{A}_{part} + c} ,$$

where Aₚₐᵣₜ is the partial area of the DSC peak; Aₜₒₜ is the total area of the peak, and c is the linearization factor

### EXAMPLES

### Example 1: Providing a HCSI of UP Grade According to the Present Invention (CSI Route)

Into a pre-dried mechanically-stirred double-jacketed 1.5 L glass stirred-tank reactor equipped with 4 baffles, a stirring shaft, a distillation equipment including a condenser (cooled by means of a cryostat) and a fraction separator, two temperature probes, connected to a thermostat (double-jacket), and to a KOH scrubber (neutralization of acidic vapors) was loaded at room temperature by cannulation under nitrogen flux chlorosulfonic acid (814.1 g), followed by chlorosulfonyl isocyanate (989 g). The mixture was heated from room temperature to reflux over 17 hours, and the reflux was maintained until gas evolution stopped. The resulting clear brown mixture obtained from such reaction comprises HCSI, heavy fractions and lights fractions, i.e., a crude HCSI mixture (I). The crude HCSI mixture (I) was pre-distilled under reduced pressure (Tₛₑₜ=90 to 120° C.; P=4 mbar abs.) in order to isolate 263 g of light fractions (T_{head}=90-107° C.) for 1.5 to 2 hours. The resulting HCSI mixture (II) was cooled to 50° C. and transferred under inert conditions into a pre-dried WFSP distillation equipment via a pre-dried double-jacketed glass addition funnel. The WFSP equipment parameters were set as follow:

$${T}_{boiler} = 80 ° C . \\ {T}_{inner condenser} = 35 ° C . \\ {T}_{funnel} = 50 ° C . \\ {P}_{WFSP} ≦ 1 mbar \\ Rotating speed := 400 rpm$$

HCSI mixture (II) (332.8 g) was introduced at a constant rate (about 120-125 g/hr) enabling the formation of a stable film at the given distillation parameters. Vapors were rapidly condensed on the inner condenser's surface, and were collected in the collection flask. The flow rate was set in order to obtain a ratio of condensed vapors/mother liquor about 6/4. The isolated pure material was extracted from the WFSP. Resulting mother liquors were re-introduced to a second WFSP distillation phase using the same distillation parameters. Another pure fraction was collected and combined with the first fraction of pure material. The distillation was stopped at this stage and the overall mass of purified HCSI (249.5 g) extracted from the WFSP was about 75% without further optimization. The residence time at the WFSP was less than 30 seconds. The isolated HCSI was solidified under inert atmosphere for 12 hours in a fridge before introducing the crystallized material into a glovebox.

### Example 2: Analysis of the HCSI of UP Grade by DSC

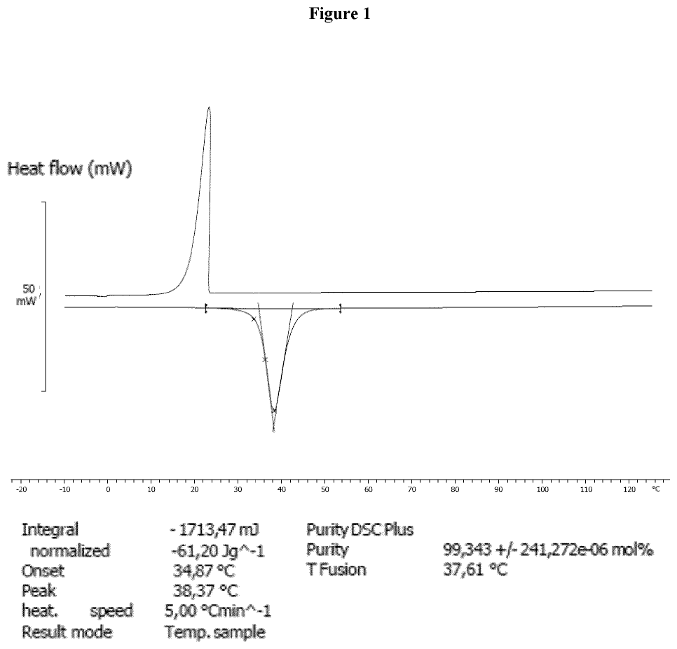

A DSC sample of the product isolated in Example 1 was prepared into a glovebox using a stainless-steel pressure-resistant crucible and a suitable press (both from Mettler Toledo). The sealed crucible containing about 10 mg of the crushed solid was taken out from the glovebox for DSC analysis. The DSC method included 4 melting and 3 crystallizations at 5° C./min between −30° C. and 150° C. under N₂ stream of 50 mL/min. (for 4 hours 12 minutes). The HCSI of UP grade as isolated and characterized by DSC showed a very sharp and symmetrical melting peak. The purity of HCSI of UP grade was determined by applying the “Purity” function of the STARe software, i.e., Version 11.00a (Mettler Toledo) software. The HCSI of UP grade sample displayed the following DSC results (see also FIG. 1):
Onset: 34.7° C.
Peak: 38.3° C.
T° fusion: 37.7° C.
Purity: about 99.3%
Normalized integral: about 62 J/g
Apex of crystallization peak: about 23° C.

Criteria for the access to the UP grade were internally defined as the following, based on cumulative observation on the HCSI of UP grade samples versus HCSI distilled in batch (Comparative Example 1):
Onset: >34° C.
Peak: >38° C.
T° fusion: >37.5° C.
Purity: >99.0%
Normalized integral: −58<x<−65 J/g
Apex of crystallization peak: >20° C.

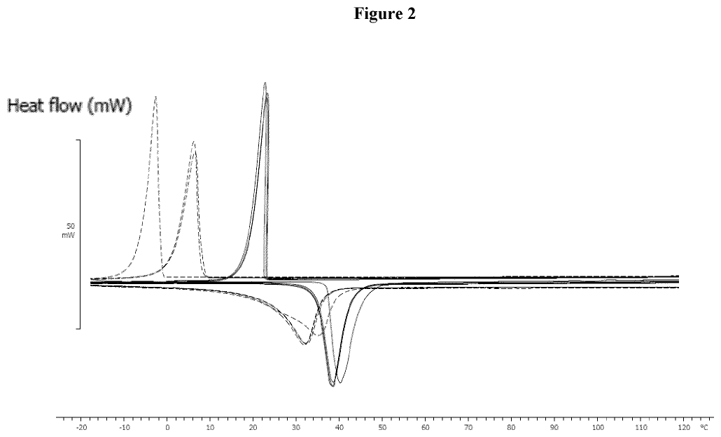

The comparison of the HCSI of UP grade (in solid lines) and the HCSI distilled in batch (in dotted lines) is shown in FIG. 2.

### Example 3: Neutralization of the HCSI of UP Grade to NH₄CSI

HCSI of UP grade (100.3 g) obtained following the protocol described in Example 1 was introduced under molten form at 60° C. into a pre-dried double-jacketed mechanically-stirred 0.1 L glass reactor equipped with 4 baffles and a condenser under inert atmosphere and heated at 60° C. The reactor was connected to a KOH scrubber to neutralize acidic vapors. Powdery NH₄Cl (24.9 g) was introduced progressively under inert atmosphere onto the molten HCSI of UP grade over 15 minutes. The mixture was heated and maintained at 75-80° C. until gas evolution stopped. A viscous colorless liquid was obtained quantitatively. Chloride analysis from the scrubber (IC, DIONEX ICS-3000) confirmed the quantitative neutralization of the released HCSI. NH₄CSI as isolated was used as such in the next Example 4.

### Example 4: Fluorination of NH₄CSI from Example 3 with NH₄F

Into a pre-dried PTFE 0.5 L mechanically-stirred reactor equipped with a 4-blades stirring shaft, 4 baffles, a PTFE condenser, an PFA-based internal tubing system connected to a thermostat (for internal heating purpose) and an insulating external layer were introduced under nitrogen stream NH₄F (38.7 g) and anhydrous EMC (283.2 g). The resulting slurry was pre-heated at 60° C. NH₄CSI (97.1 g) prepared in Example 3 was pre-heated at 60° C. and was introduced under molten form at constant flow rate. After the addition, the mixture was heated from 60° C. to 84° C. for 1 hour, the temperature was maintained for 3 hours more at 84° C. before cooling to room temperature. The suspension was transferred into a Büchner-type filter equipped with a 0.22 μm PTFE membrane under nitrogen stream. The emptied reactor was washed with additional EMC (164.2 g), further used to wash the solid cake. The resulting combined filtrate (563 g) showed a yield of 91.3% in NH₄FSI (76 g), as measured by ¹⁹F NMR (Bruker Avance 400 NMR). The following Table 1 shows IC results (DIONEX ICS-3000) of a reduced amount of most of main impurities (F⁻, Cl⁻, SO₄²⁻, FSO₃⁻) and an absence of additional impurities.

TABLE 1
IC results of impurities of NH₄FSI in EMC

| | F⁻ | Cl⁻ | SO₄²⁻ | FSO₃_{^{−}} | Other |
|---|---|---|---|---|---|
| Sample | (ppm) | (ppm) | (ppm) | (ppm) | impurities |
| NH₄FSI in EMC | 361 | 24 | <5 | 405 | No |
| (filtrate) | | | | | |

### Example 5: Precipitation of Crude NH₄FSI in Solid

The filtrate containing NH₄FSI in EMC prepared in Example 4 was transferred into a magnetically-stirred PTFE flask. Water (14.6 g) and 25% aqueous NH₄OH (0.21 g) were added to the mixture stirred at room temperature for 1 hour. This solution was concentrated under reduced pressure in order to obtain a 60 wt. % solution of NH₄FSI in EMC. The resulting concentrate was transferred into a pre-dried mechanically-stirred double-jacketed 0.3 L glass reactor equipped with 4 baffles and a condenser. Dichloromethane (DCM) (74.2 g) was introduced using a pump over 1 hour, the mixture was then cooled to 0° C. over 1 hour. DCM (73.3 g) was again dosed over 1 hour, the resulting mixture was maintained at 0° C. for 1 hour more. The resulting suspension was transferred into a Buchner-type filter equipped with a 0.22 μm PTFE membrane under nitrogen stream. The resulting solid cake composed of crude NH₄FSI was washed with DCM (78.9 g). The resulting solid was dried under reduced pressure. The overall non-optimized precipitation yield of solid crude NH₄FSI as isolated was 85.2%.

### Example 6: Purification of Precipitated Crude NH₄FSI

The resulting solid NH₄FSI (64.7 g) was transferred into a pre-dried mechanically-stirred double-jacketed 0.3 L glass reactor equipped with 4 baffles and a condenser. 291 g of 2,2,2-trifluoroethanol (TFE) was added subsequently. The overhead stirrer was set at 350 rpm. The temperature of the solution was set to 60° C. to ensure a complete dissolution of NH₄FSI in TFE. Then, 291 g of 1,4-dioxane was added dropwise to the reactor for 3 hours. After completion of the 1,4-dioxane addition, the solution temperature was kept at 60° C. for additional 3 hours. The resulting slurry was naturally cooled down to room temperature in about 3 hours, and the stirring was maintained for about 12 hours. The slurry was filtrated using a 0.22 μm PTFE membrane to collect the solid NH₄FSI. The collected solid cake was washed with 131 g of 1,4-dioxane. The 156.7 g of the collected wet solid was dried using a rotary evaporator under 70° C. at 20 mbar abs. until there was no more solvent evaporation to afford 72.7 g of a white solid, being a crystalized solvate of NH₄FSI (denoted as NH₄FSI-S1) comprising 80.5 wt. % of NH₄FSI and 19.5 wt. % of 1,4-dioxane, as confirmed by 19F-NMR (Bruker Avance 400 NMR). The purification yield was 90.4%. The process was carried out a second time on 70.1 g of the product recovered from the first precipitation, using the following amounts of chemicals: 255.1 g of TFE, 242.4 g of 1,4-dioxane for the crystallization and 132 g of 1,4-dioxane for the washing. After drying, 66.6 g of a white solid was obtained, being a crystalized solvate of NH₄FSI (denoted as NH₄FSI-S2) comprising 79.6 wt. % of NH₄FSI and 20.4 wt. % of 1,4-dioxane. as confirmed by 19F-NMR (Bruker Avance 400 NMR). The second purification yield was 94%.

The following Table 2 shows IC (DIONEX ICS-3000) results of the crude NH₄FSI and the products, i.e., NH₄FSI solvates (NH₄FSI-S1 and NH₄FSI-S2) obtained after the first purification and the second purification.

TABLE 2
IC results of the crude NH₄FSI and NH₄FSI solvates S1 and S2

| | F⁻ | Cl⁻ | SO₄²⁻ | FSO₃_{^{−}} | Other |
|---|---|---|---|---|---|
| Sample | (ppm) | (ppm) | (ppm) | (ppm) | impurities |
| Crude NH₄FSI | 1474 | 87 | 119 | 2815 | No |
| NH₄FSI-S1 | 33 | 2 | 87 | 21 | No |
| NH₄FSI-S2 | 19 | N.D. | 56 | N.D. | No |

* N.D. Non-Detected

### Example 7: Lithiation of the Purified NH₄FSI

65 g of NH₄FSI-S2 obtained in Example 6 was dissolved in 217 g of butyl acetate and then 48.2 g of a 25 wt. % aqueous solution of LiOH. H₂O was added. The biphasic mixture as obtained was stirred during 5 hours at room temperature, and then decanted. The organic phase was recovered and put into a thin-film evaporator at 60° C. under reduced pressure (0.1 bar abs.). The purity of the obtained lithium bis(fluorosulfonyl)imide (LiFSI) was above 99.99 mol. % as determined by 19F-NMR (Bruker Avance 400 NMR): chlorine and fluorine contents were below 20 ppm, and metal elements contents were below 3 ppm, with no other impurities such as SO₄²⁻ and FSO₃⁻ detected by IC (DIONEX ICS-3000).

### Comparative Example 1: Preparation of HCSI Using Batch Distillation

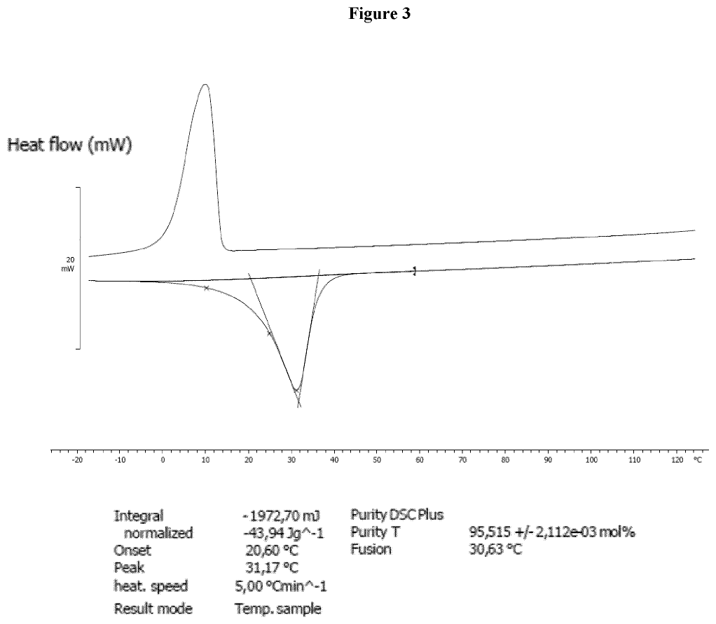

Into a pre-dried mechanically-stirred double-jacketed 1.5 L glass stirred-tank reactor equipped with 4 baffles, a stirring shaft, a distillation equipment including a condenser (cooled by means of a cryostat) and a fraction separator, two temperature probes, connected to a thermostat (double-jacket), and to a KOH scrubber (neutralization of acidic vapors) was loaded at room temperature by cannulation under nitrogen flux chlorosulfonic acid (868.8 g), followed by chlorosulfonyl isocyanate (1011.9 g). The mixture was heated from room temperature to reflux over 17 hours, and the reflux was maintained until gas evolution stopped. The resulting clear brown HCSI mixture (I) comprises HCSI, heavy fractions and lights fractions. The mixture was pre-distilled under reduced pressure (Tₛₑₜ=95 to 120° C.; P=6-7 mbar abs.) to isolate 330.1 g of light fractions (T_{head}=90-115° C.) after about 2 hours. The resulting HCSI mixture (II) was further distilled in the initial vessel to isolate two HCSI fractions (Tₛₑₜ=120 to 145° C.; T_{head}=115 to 118° C., P=about 6-7 mbar abs) after about 5 to 6 hours, during which traces of light fractions appeared, in addition to the heavy fractions and HCSI, due to the additional thermal degradation. The resulting fractions were combined to give 896.3 g of distilled HCSI. DSC analysis of HCSI distilled in batch is shown in FIG. 3.

### Comparative Example 2: WFSP Distillation of the HCSI Previously Distilled in Batch

Distilled HCSI obtained in Comparative Example 1 was transferred at 50° C. under inert conditions into a pre-dried WFSP distillation equipment via a pre-dried double-jacketed glass addition funnel. The WFSP equipment parameters were set as follow:
T_{boiler}: 80° C.
T_{inner condenser}: 35° C.
T_{funnel}: 50° C.
P_{WFSP}; less than 1 mbar abs.
Rotating speed: 400 rpm.

Distilled HCSI (122.7 g) was introduced at a constant rate (about 120-125 g/hr) enabling the formation of a stable film at the given distillation parameters. Vapors were rapidly condensed on the inner condenser's surface, and were collected in the collection flask. The flow rate was set in order to obtain a ratio of condensed vapors/mother liquors about 8/2. The isolated material was extracted from the WFSP. The distillation was stopped at this stage, the overall mass of distilled HCSI (101.2 g) extracted from the WFSP was about 82% without further optimization. The isolated HCSI was solidified under inert atmosphere for 12 hours in a fridge before careful introduction of the crystallized material into a glovebox for DSC analysis. The results can be observed on FIG. 3. The shape of the melting peak was broad and unsymmetrical, with a melting temperature of 30.2° C. The molar purity was assessed about 95.5%. The comparison of HCSI of UP grade and HCSI distilled in batch is shown in FIG. 2.

### Comparative Example 3: Direct Fluorination of the HCSI Distilled in Batch Distillation Using NH₄F

Into a pre-dried PTFE 0.5 L mechanically-stirred reactor equipped with a 4-blades stirring shaft, 4 baffles, a PTFE condenser, an PFA-based internal tubing system connected to a thermostat (for internal heating purpose) and an insulating external layer were introduced under nitrogen stream NH₄F (77.1 g) and anhydrous EMC (307.9 g). The resulting slurry was pre-heated at 60° C. HCSI (97.1 g) obtained according to Comparative Example 1 was pre-heated at 60° C. and was introduced under molten form at constant flow rate. After the addition, the mixture was maintained for 3 hours at 84° C. before cooling to room temperature. The suspension was transferred into a Büchner-type filter equipped with a 0.22 μm PTFE membrane under nitrogen stream. The emptied reactor was washed with additional EMC (164.7 g), further used to wash the solid cake. The resulting combined filtrate (474.7 g) showed a yield of 93% in NH₄FSI (83.6 g), as measured by 19F NMR (Bruker Avance 400 NMR). IC (DIONEX ICS-3000) results showed an impurity profile superior to Example 4, with higher content of main impurities (F⁻, Cl⁻, SO₄²⁻, NH₂SO₃⁻, FSO₃⁻) and the presence of additional impurities.

### Comparative Example 4: Neutralization of the HCSI Distilled in Batch to NH₄CSI

HCSI (100.7 g) obtained according to Comparative Example 1 was introduced under molten form at 60° C. into a pre-dried double-jacketed mechanically-stirred 0.1 L glass reactor equipped with 4 baffles and a condenser under inert atmosphere and heated at 60° C. The reactor was connected to a KOH scrubber to neutralize acidic vapors. NH₄Cl (24.9 g) in powder was introduced progressively under inert atmosphere onto molten HCSI UP over 15 minutes. The mixture was heated and maintained at 75-80° C. until gas evolution stopped. A viscous colorless liquid was obtained quantitatively. Chloride analysis from the scrubber (IC, DIONEX ICS-3000) confirmed quantitative neutralization of the released HCSI. NH₄CSI as isolated was used as such in the next example.

### Comparative Example 5: Fluorination of the NH₄CSI from Comparative Example 3 by NH₄F

NH₄CSI (98.1 g) obtained in Comparative Example 4 was submitted to the identical fluorination conditions as described in Example 4, to provide a combined filtrate (404.8 g) showing a yield of 92.2% in NH₄FSI (77.6 g), as measured by 19F NMR. IC (DIONEX ICS-3000) results showed an increased amount of most of the main impurities (F⁻, Cl⁻, SO₄²⁻, FSO₃⁻) as shown in the below Table 3 in comparison with Example 4 and the presence of additional impurities.

TABLE 3
IC results of NH₄FSI

| | F⁻ | Cl⁻ | SO₄²⁻ | FSO₃_{^{−}} | Others |
|---|---|---|---|---|---|
| Sample | (ppm) | (ppm) | (ppm) | (ppm) | impurities |
| NH₄FSI in EMC | 420 | 26 | 9 | 318 | Yes |
| (filtrate) | | | | | |

### Comparative Example 6: Precipitated of Crude Solid NH₄FSI

The filtrate prepared in Comparative Example 5 was submitted to successive steps following strictly the operating conditions from Examples 5 and 6, to provide a precipitated crude NH₄FSI in white solid. The overall precipitation yield was comparable to Example 5 without optimization, and the purification yields were similarly comparable to the first and second purifications from Example 6. After drying, 68 g of a white solid was obtained, being a crystalized solvate of NH₄FSI (denoted as NH₄FSI-S2) comprising 80.4 wt. % of NH₄FSI and 19.6 wt. % of 1,4-dioxane, as confirmed by 19F-NMR (Bruker Avance 400 NMR).

The following Table 4 shows IC (DIONEX ICS-3000) results of the comparative crude NH₄FSI and the comparative NH₄FSI solvates obtained after a first purification and a second purification.

TABLE 4
IC results of comparative crude NH4FSI and NH4FSI solvates
S1 and S2

| | F⁻ | Cl⁻ | SO₄²⁻ | FSO₃_{^{−}} | Other |
|---|---|---|---|---|---|
| Sample | (ppm) | (ppm) | (ppm) | (ppm) | impurities |
| Crude NH4FSI | 2481 | 90 | 89 | 2815 | Yes |
| NH₄FSI-S1 | 85 | 18 | 74 | 80 | Yes |
| NH₄FSI-S2 | 31 | <5 | 39 | 11 | Yes |

### Comparative Example 7: Lithiation of the NH₄FSI as Purified

60 g of NH₄FSI-S2 obtained in Comparative Example 6 was dissolved in 200 g of butyl acetate. Subsequently, 44.5 g of a 25 wt. % aqueous solution of LiOH. H₂O was added. The biphasic mixture as obtained was stirred during 5 hours at room temperature, and then decanted. The organic phase was recovered and put into a thin-film evaporator at 60° C. under reduced pressure (0.1 bar abs.). The purity of the obtained lithium bis(fluorosulfonyl)imide (LIFSI) was above 99.99 mol. % as determined by 19F-NMR (Bruker Avance 400 NMR); chlorine and fluorine contents were below 40 ppm; other impurities contents such as SO₄²⁻ and FSO₃⁻ were below 20 ppm by IC (DIONEX ICS-3000), and metal elements contents were below 3 ppm (ICP analysis).

It was clearly demonstrated in the Examples that the HCSI of UP grade manufactured according to the process of the present invention resulted in an increased performance in the subsequent steps to finally produce a higher purity of LiFSI in high yield, and notably HCSI was obtained under milder conditions, including temperature conditions and residence time required to purify the HCSI of UP grade.

In addition, the inventor also found that using the HCSI of UP grade obtained according to the present process to synthesize a LiFSI reduces the need for purification, while causing an improved impurity profile of the final LiFSI without compromising the yield. The reduced level of impurities obtained before the fluorination step reduces the overall environmental impact of the whole LiFSI process as the need for purification step(s) are reduced. Finally, the improved quality of the final LiFSI product generates a superior performance in the application of this product in lithium-ion secondary batteries

1. A process for manufacturing a bis(chlorosulfonyl)imide (HCSI) of ultra-pure (UP) grade comprising the steps of:
(i) providing a crude HCSI mixture (I) comprising HCSI, heavy fractions and light fractions;
(ii) removing the light fractions from the crude HCSI mixture (I) so as to obtain a HCSI mixture (II);
(iii) transferring the HCSI mixture (II) to a thin-film evaporator; and
(iv) distilling the HCSI mixture (II) to isolate the HCSI of UP grade, wherein the HCSI of UP grade presents a purity of at least 99.0 mol. % with respect to the total number of moles of HCSI, as determined by differential scanning calorimetry (DSC) according to ASTM E928-19.

2. The process according to claim 1, wherein the crude HCSI mixture (I) is obtained from:
reacting chlorosulfonic acid and chlorosulfonyl isocyanate, or
reacting sulfamic acid, chlorosulfonic acid and thionyl chloride.

3. The process according to claim 1, wherein the purity of the HCSI of UP grade is at least 99.3 mol. % with respect to the total number of moles of HCSI.

4. The process according to claim 1, wherein the thin-film evaporator is a short-path thin-film evaporator, a wiped-film short-path (WFSP) evaporator (with or without external condenser), or a falling-film evaporator.

5. The process according to claim 1, wherein the distillation step (iv) is implemented at a temperature of from 60 to 120° C.

6. The process according to claim 1, wherein the distillation step (iv) is implemented at a pressure of 10 mbar abs. or less.

7. The process according to claim 1, wherein the distillation step (v) is implemented for 5 min. or less.

8. The process according to claim 1, wherein the light fractions comprise chlorosulfonic acid, chlorosulfonyl isocyanate, and thionyl chloride.

9. The process according to claim 1, wherein the heavy fractions comprise by-products from the reaction mixture including dimers, trimers, and other oligomers.

10. An HCSI of UP grade obtainable from a process according to claim 1, wherein the HCSI presents a purity of at least 99.0 mol. % with respect to the total number of moles of HCSI, determined by differential scanning calorimetry (DSC) according to ASTM E928-19.

11. (canceled)

12. A process for manufacturing a lithium bis(fluorosulfonyl)imide (LiFSI), comprising the preparation of an HCSI of UP grade according to claim 1.

13. The process according to claim 12 comprising the steps of:
(i) providing an HCSI of UP grade by a process according to claim 1;
(ii) fluorinating the HCSI of UP grade with a fluorinating agent to form an ammonium bis(fluorosulfonyl)imide (NH₄FSI); and
(iii) optionally purifying the NH₄FSI obtained from the step (ii); and
(iv) lithiating the NH₄FSI optionally in a form of a solvate with at least one solvent S₂, with a lithiating agent to form a LiFSI.

14. The process according to claim 13, wherein in step (iv), the NH₄FSI is a solvate, in a crystallized form, comprising:
50 to 98 wt. %, of the NH₄FSI salt, and
2 to 50 wt. %, of solvent S₂, which is selected from the group consisting of cyclic and acyclic ethers.

15. The process according to claim 13, wherein step (iii) comprises:
(iii₁) dissolving the NH₄FSI from step (ii) in at least one solvent S₁;
(iii₂) crystallizing NH₄FSI from step (iii₁) by means of at least one solvent S2; and
(iii₃) separating the NH₄FSI salt from at least part of the solvents S₁ and S₂ to prepare a NH₄FSI solvate.
